(19) United States
(12) Patent Application Publication
(10) Pub. No.: US 2002/0068102 A1
(43) Pub. Date: Jun. 6, 2002

(54) Reducing cellular damage in the human body

(76) Inventors: Chen Xing Su, West Jordan, UT (US); Claude Jarakae Jensen, Cedar Hills, UT (US); Stephen P. Story, Alpine, UT (US)

Correspondence Address: Michael F. Krieger, KIRTON & McCONKIE, Suite 1800, 60 East South Temple, Salt Lake City, UT, 84111, US

(21) Appl. No.: 09/997,588

(22) Filed: Nov. 29, 2001

Related U.S. Application Data
(60) Provisional application No. 60/250,648, filed on Dec. 1, 2000.
(60) Provisional application No. 60/251,417, filed on Dec. 5, 2000.

(51) Int. Cl. A61K 35/78

(52) U.S. Cl. 424/765; 424/777

(57) ABSTRACT

Providing and using a dietary supplement to reduce cellular damage within the human body. The dietary supplement includes reconstituted Morinda citrifolia fruit juice from pure juice puree of French Polynesia, and may include other natural juices, such as a natural grape juice concentrate, a natural blueberry juice concentrate, and/or another natural juice concentrate. The dietary supplement includes a combination of compounds that work at the cellular level to increase the positive functionality of cells in the body, including cell regeneration and cell function, to increase the ability of cells to absorb and utilize nutrients, such as vitamins and minerals, and to. Furthermore, the dietary supplement scavenges lipid hydroperoxides and superoxide anion free radicals within the body to reduce cellular damage.

### RELATED APPLICATIONS

This application claims priority to U.S. Provisional Patent Application Serial No. 60/250,648, filed Dec. 1, 2000, entitled “ANTIOXIDANT ACTIVITY OF TAHITIAN NONI JUICE,” and to U.S. Provisional Patent Application Serial No. 60/251,417, filed Dec. 5, 2000, entitled “ANTIOXIDANT STUDIES ON TAHITIAN NONI JUICE.”

### BACKGROUND OF THE INVENTION

1. Field of the Invention

The present invention relates to reducing cellular damage in the human body. In particular, the present invention relates to the use of a dietary supplement to scavenge lipid hydroperoxides and superoxide anion free radicals within the body.

3. Background and Related Art

Natural cell processes exist within the human body that use oxygen and produce toxins that are commonly referred to as “free radicals.” These free radicals may be highly reactive oxidizing substances that attach to and attack carbohydrates, deoxyribonucleic acid (“DNA”), enzymes, fats, and/or proteins within the body. The free radicals typically interfere with cellular function and reproduction, and may cause dysfunction and/or death of cells, tissues, and organs within the body.

While a natural defense mechanism exists to reduce the cellular damage caused by the free radicals, the defense mechanism may become increasingly inefficient as the body ages. As such, damage caused by the free radicals has been implicated in several age-associated diseases, such as Alzheimer's disease, cancer, diabetes, heart disease, macular degeneration, and Parkinson's disease. In fact, suggestions have even been made that the damage caused by the free radicals may be an integral factor in the aging process of the human body.

The amount of free radicals produced within the body is typically increased as the individual is exposed to cigarette smoke or various other toxins, such as mercury. Furthermore, the production of the free radicals is typically enhanced by exercise, since exercise instigates a need for oxygen within the body.

One technique currently used to reduce cellular damage caused by the free radicals includes the consumption of ascorbic acid, commonly known as “Vitamin C”. Studies have indicated that regular consumption of Vitamin C may provide desirable benefits to individuals with coronary artery disease, diabetes, high blood pressure, high cholesterol, and high blood levels of homocysteine.

Another technique used to reduce the cellular damage includes the regular consumption of biofavonoids, such as a mixture of catechins, phenolic acid, proan, and thocyanidins. One such mixture is made from a highly bioactive substance called proanthocyanidins, obtained from the bark of the Maritime Pine in France and is the active ingredient in a product known as Pycnolgeol®. According to some reports, this mixture of biofavonoids may be absorbed in the skin and retained for as long as 72 hours to neutralize free radicals. It may improve circulation and may strengthen the immune system.

Another technique used to reduce cellular damage includes the consumption of grape seed extract, which has been said to strengthen and protect living tissues. Reports have indicated that grape seed extract may strengthen blood vessels, improve the skin, and aid circulation. Furthermore, grape seed extract may be advantageous in defeating the hormone dihydrotesterone (“DHT”), which prevents the hair follicle growth cycle, thereby stimulating healthy hair growth.

While reports have claimed that various substances may be used to reduce cellular damage caused by the free radicals, contradictory results have been obtained that cause researchers to continue searching for answers. Accordingly, it would be an improvement in the art to augment or even replace the substances currently used with other substances to further reduce cellular damage in the human body.

### SUMMARY OF THE INVENTION

The present invention relates to reducing cellular damage in the human body. In particular, the present invention relates to the use of a dietary supplement to scavenge lipid hydroperoxides and superoxide anion free radicals within the body.

Implementation of the present invention takes place in association with a dietary supplement that is processed from the fruit of the Indian Mulberry plant, scientifically known as Morinda citrifolia L. In one implementation, the dietary supplement includes reconstituted Morinda citrifolia fruit juice from pure juice puree of French Polynesia. The supplement may also include other natural juices, such as a natural grape juice concentrate, a natural blueberry juice concentrate, and/or another natural juice concentrate. In one implementation, the dietary supplement is not processed from dried or powdered Morinda citrifolia, rather liquid is extracted from the fruit of the Morinda citrifolia and used to create the dietary supplement. One implementation of the dietary supplement is referred to as “Tahitian Noni®” and may be obtained from Morinda, Inc., which has a principal place of business located at 5152 N. Edgewood Dr. #100, Provo, Utah, 84604.

Use of the dietary supplements described herein include scavenging lipid hydroperoxides and superoxide anion free radicals within the body, thereby reducing cellular damage in the human body. The dietary supplements include a combination of compounds that work at the cellular level to increase the positive functionality of cells in the body, including cell regeneration and cell function. The combination of compounds increases the ability of cells within the body to absorb and utilize nutrients such as vitamins and minerals, and stimulates the production of T-cells within the immune system. The T-cells are a type of lymphocyte or white blood cell that lead the attack against infections within the body, end the immune response, and/or kill cancer cells and/or cells infected with a virus.

In one implementation, one ounce of the dietary supplement is consumed per day to reduce toxins produced by natural cell processes with in the human body. Alternatively, consumption amounts may include more than one ounce per day or less than one ounce per day. Furthermore, the dietary supplement may be taken in the morning and/or before meals.

While the methods and processes of the present invention have proven to be particularly useful in the area of scavenging lipid hydroperoxides and superoxide anion free radicals within the body, those skilled in the art can appreciate that the methods and processes of the present invention can be used in a variety of different applications and in a variety of different ways to reduce cellular damage within the body.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### DETAILED DESCRIPTION OF THE INVENTION

The present invention relates to reducing cellular damage in the human body. In particular, the present invention relates to the use of a dietary supplement to scavenge lipid hydroperoxides and superoxide anion free radicals within the body.

The following disclosure of the present invention is grouped into two subheadings, namely “Dietary Supplement” and “Reducing Cellular Damage.” The utilization of the subheadings is for convenience of the reader only and is not to be construed as limiting in any sense.

### Dietary Supplement

Embodiments of the present invention take place in association with a dietary supplement that is processed from the fruit of the Indian Mulberry plant, scientifically known as Morinda citrifolia L. The plant is an evergreen tree or shrub that is typically found in open tropical coastal regions, such as in Asia, Australia, and in islands of the Pacific Ocean. It is a member of the coffee family and typically grows in open lowlands, often on or along lava flows, and at edges of forests. The straight trunk, large, green, elliptical leaves, white tubular flowers, and ovoid, yellow fruit readily identify the Morinda citrifolia.

The plant may be cultivated in gardens and has been naturalized in both moist and dry areas from sea level to about 1,300 feet above sea level. In particular, the Morinda citrifolia flourishes in the lush and unspoiled lands of French Polynesia, which includes Tahiti, and grows particularly large and lush in French Polynesia because of the ideal climate and soil conditions, and because the islands are generally still in their pristine condition. A mature plant may reach heights of 15 to 20 feet tall and may bear fruit throughout the year.

The Morinda citrifolia typically includes coarse branches that are angular in cross section. The branches are thick and may be conspicuously marked with leaf scars. The leaves are ovate, thick, deeply veined, short-stemmed, green, and shiny, and may grow to lengths of over eight inches.

Small white flowers of the Morinda citrifolia are born on globose, fused heads to form an inflorescence that is typically about an inch in diameter. While each of the heads produces many flower buds, typically only one or two of the buds open on a particular head during a given period of time. The flowers typically include five to seven lobed corollas that are often about one-third of an inch long in size. The flowers are closely packed and appear in various stages of development on the head. For example, green buds may be present near the apex of a particular head, while older flowers near the base of the head may have already opened and closed. Before the last flower near the apex of a head has bloomed, small yellowish-green fruitlets replace the flowers.

When the fruit of the Morinda citrifolia is mature, it is typically several inches long. Generally, the fruit is elongate in shape and includes a warty appearance that is caused by being marked with the polygonal outlines of the uneven individual fruitlet growth patterns. A pit, left as a scar from the corolla, is located within each of the polygonal outlines. If picked green, the fruit typically rots. As such, it is typically advantageous to allow the fruit to mature and ripen prior to picking.

Once the fruit of the Morinda citrifolia has matured, the skin is typically a whitish color and the flesh is a whitish-yellow color. The mature fruit is generally the size of a potato and resembles a small breadfruit. When the fruit is ripe or overripe, the skin becomes translucent and the flesh becomes soft and provides a foul odor and/or taste. The seeds or kernels of the fruit are generally triangular in shape and are reddish-brown in color. An air sac, attached to one end of a seed, allows a detached seed to be buoyant.

The Morinda citrifolia is rich in natural ingredients. For example, the natural ingredients include: (from the leaves) alanine, anthraquinones, arginine, ascorbic acid, aspartic acid, calcium, beta-carotene, cysteine, cystine, glycine, glutamic acid, glycosides, histidine, iron, leucine, isoleucine, methionine, niacin, phenylalanine, phosphorus, proline, resins, riboflavin, serine, beta-sitosterol, thiamine, threonine, tryptophan, tyrosine, ursolic acid, and valine; (from the flowers) acacetin-7-o-beta-d(+)-glucopyranoside, 5,7-dimethyl-apigenin-4′-o-beta-d(+)-galactopyranoside, and 6,8-dimethoxy-3-methylanthraquinone-1-o-beta-rhamnosyl-glucopyranoside; (from the fruit) acetic acid, asperuloside, butanoic acid, benzoic acid, benzyl alcohol, 1-butanol, caprylic acid, decanoic acid, (E)-6-dodeceno-gamma-lactone, (Z,Z,Z)-8,11,14-eicosatrienoic acid, elaidic acid, ethyl decanoate, ethyl hexanoate, ethyl octanoate, ethyl palmitate, (Z)-6-(ethylthiomethyl) benzene, eugenol, glucose, heptanoic acid, 2-heptanone, hexanal, hexanamide, hexanedioic acid, hexanoic acid (hexoic acid), 1-hexanol, 3-hydroxy-2-butanone, lauric acid, limonene, linoleic acid, 2-methylbutanoic acid, 3-methyl-2-buten-1-ol, 3-methyl-3-buten-1-ol, methyl decanoate, methyl elaidate, methyl hexanoate, methyl 3-methylthio-propanoate, methyl octanoate, methyl oleate, methyl palmitate, 2-methylpropanoic acid, 3-methylthiopropanoic acid, myristic acid, nonanoic acid, octanoic acid (octoic acid), oleic acid, palmitic acid, potassium, scopoletin, undecanoic acid, (Z,Z)-2,5-undecadien-1-ol, and vomifol; (from the roots) anthraquinones, asperuloside (rubichloric acid), damnacanthal, glycosides, morindadiol, morindine, morindone, mucilaginous matter, nor-damnacanthal, rubiadin, rubiadin monomethyl ether, resins, soranjidiol, sterols, and trihydroxymethyl anthraquinone-monomethyl ether; (from the root bark) alizarin, chlororubin, glycosides (pentose, hexose), morindadiol, morindanigrine, morindine, morindone, resinous matter, rubiadin monomethyl ether, and soranjidiol; (from the wood) anthragallol-2,3-dimethylether; (from the tissue culture) damnacanthal, lucidin, lucidin-3-primeveroside, and morindone-6beta-primeveroside; and (from the plant) alizarin, alizarin-alpha-methyl ether, anthraquinones, asperuloside, hexanoic acid, morindadiol, morindone, morindogenin, octanoic acid, and ursolic acid.

The fruit of the Morinda citrifolia includes health-enhancing enzymes that, for example, aid in easing inflammation, calming feelings of anxiety, supporting weight management, and promoting circulatory health in humans. The Morinda citrifolia is an adaptogenic herb that supports balanced body systems by responding to the body's need for stimulation or relaxation. Embodiments of the present invention relate to the creation and utilization of a dietary supplement that includes fruit juice from Morinda citrifolia.

In one embodiment, the dietary supplement includes reconstituted Morinda citrifolia fruit juice from pure juice puree of French Polynesia. The supplement may also include other natural juices, such as a natural grape juice concentrate, a natural blueberry juice concentrate, and/or another natural juice concentrate. In one embodiment, the dietary supplement is not processed from dried or powdered Morinda citrifolia, rather liquid is extracted from the fruit of the Morinda citrifolia and used to create the dietary supplement, as will be discussed below. In one embodiment, the dietary supplement is referred to as “Tahitian Noni®” and may be obtained from Morinda, Inc., which has a principal place of business located at 5152 N. Edgewood Dr. #100, Provo, Utah, 84604.

While the following discussion provides a representative process for creating a dietary supplement that may be used to reduce cellular damage in accordance with the present invention, those skilled in the art of creating dietary supplements shall appreciate that embodiments of the present invention embrace other methods and/or processes may be used in place of or in addition to those disclosed below to create a dietary supplement for reducing cellular damage in accordance with the present invention.

In one embodiment, the fruit of the Morinda citrifolia is harvested, either by hand or by mechanical equipment, when it is at least one inch long and up to 12 inches in diameter. At this time, the fruit generally has a color ranging from a dark green through a yellow-green up to a white color, and gradations of color in between. The fruit is thoroughly cleaned after being harvested and before being processed.

The fruit is allowed to ripen or age from 0 to 14 days by being placed on equipment so that the ripening fruit is prevented from contacting the ground. The fruit is typically covered with a cloth or netting material during aging, but can be aged without being covered. When ready for further processing the fruit is light in color, such as a light green, a light yellow, a white, or a translucent color. The fruit is inspected for spoilage and/or for excessively green color and hard firmness. Spoiled and hard green fruit is separated from the acceptable aged fruit.

The acceptable aged fruit is typically placed in plastic lined containers for further processing and transport. While the containers of fruit may be held from 0 to 30 days, most containers are generally held for 7 to 14 days before processing. The containers can optionally be stored under refrigerated conditions prior to further processing.

The fruit is unpacked from the storage containers and is processed through a manual or mechanical separator. The seeds and peel are separated from the juice and pulp. The juice and pulp may be packaged into containers for storage and transport. The containers may be stored in refrigerated, frozen, or room temperature conditions. Alternatively, the juice may be immediately processed into a finished juice product.

Filtering equipment may be used to remove pulp from the juice. The filtering equipment may include a centrifuge decanter, a screen filter with a size from 1 micron up to 2000 microns (in one embodiment it is more preferably less than 500 microns), a filter press, reverse osmosis filtration, and/or any other standard commercial filtration devices. The operating filter pressure may range from 0.1 psig up to about 1000 psig. The flow rate may range from 0.1 g.p.m. up to 1000 g.p.m., and in one embodiment more preferably between 5 and 50 g.p.m. The wet pulp may be washed and filtered at least once to remove any juice from the pulp. The wet pulp typically has a fiber content of 10 to 40 percent by weight and may be pasteurized at a minimum temperature of 181° F. (83° C.) and then packed in drums for further processing or made into a high fiber product.

The Morinda citrifolia juice and puree are typically blended in a homogenous blend, after which they are mixed with other ingredients, such as flavorings, sweeteners, nutritional ingredients, botanicals, extracts, and/or colorings. For example, flavorings may include, but are not limited to, artificial and/or natural flavor or ingredients that contribute to palatability. Examples of sweeteners include, but are not limited to, natural sugars derived from corn, sugar beet, sugar cane, potato, tapioca, or other starch-containing sources that are chemically or enzymatically converted to crystalline chunks, powders, and/or syrups, or other sweeteners, including artificial or high intensity sweeteners, some of which are aspartame, sucralose, stevia, saccharin, etc. Examples of nutritional ingredients include vitamins (e.g., A, B1 through B12, C, D, E, Folic Acid, Pantothenic Acid, Biotin, etc.), minerals and/or trace elements (e.g., calcium, chromium, copper, cobalt, boron, magnesium, iron, selenium, manganese, molybdenum, potassium, iodine, zinc, and/or phosphorus), herbs and/or botanical extracts (e.g., alfalfa grass, bee pollen, chlorella powder, Dong Quai powder, Ecchinacea root, Gingko Biloba extract, Horsetail herb, Shitake mushroom, spirulina seaweed, and/or grape seed extract), bioactive chemicals (e.g., caffeine, ephedrine, Lcarnitine, creatine, and/or lycopene), and/or compounds.

The finished juice product is typically heated and pasteurized at a minimum temperature of 181° F. (83° C.) or higher, such as up to 212° F. (100° C.). The product is filled and sealed into a final container of plastic, glass, or another suitable material that withstands the processing temperatures. The containers are maintained at the filling temperature or may be cooled rapidly and then placed in a shipping container. The shipping containers are typically wrapped with a material and in a manner to maintain or control the temperature of the product in the final containers.

### Reducing Cellular Damage

As provided above, embodiments of the present invention relate to reducing cellular damage in the human body. Natural cell processes exist that use oxygen and produce toxins, known as “free radicals.” The free radicals are chemical species that possess an unpaired electron and are highly reactive oxidizing substances that attach to and attack carbohydrates, deoxyribonucleic acid (“DNA”), enzymes, fats, and/or proteins within the body. The free radicals are produced continuously in cells either as accidental by-products of metabolism or deliberately during, for example, phagocytosis. The free radicals typically interfere with cellular function and reproduction, and may cause dysfunction and/or death of cells, tissues and organs within the body.

While natural defense mechanisms exist to reduce the cellular damage caused by the free radicals, the defense mechanism may become increasingly inefficient as the human body ages. As such, damage caused by the free radicals has been implicated in several age-associated diseases, such as Alzheimer's disease, cancer, diabetes, heart disease, macular degeneration, and Parkinson's disease. In fact, suggestions have even been made that the damage caused by the free radicals may be an integral factor in the aging process of the human body.

The amount of free radicals produced within the body is typically increased as the individual is exposed to cigarette smoke or various other toxins, such as mercury. Furthermore, the production of free radicals is typically enhanced by exercise, since exercise instigates a need for oxygen within the body.

Excessive production of free radical-mediated oxidative alteration of fatty acids, also known as lipid peroxidation, leads to damage of cellular structure, enzymes, and/or tissues. Reactions occur in the body to form hydrogen peroxide and the highly toxic hydroxyl radical. A superoxide anion is converted to a hydroxyl radical and interacts with nitric oxide to form peroxinitrite, which degrades to form a hydroxyl radical. Peroxy radicals typically remove hydrogen from lipids, such as polyunsaturated fatty acids, resulting in a formation of lipid hydroperoxides and further propagate radical pathways by regeneration of alkyl radicals. Hydroperoxides have direct toxic effects for endothelial cells and degrade to form the hydroxyl radical. Hydroperoxides also form stable aldehydes, such as malondialdehyde (MDA), which damage membranes by facilitating the formation of protein cross-links and other end products.

Hydroperoxyeicosatetraenoic acids (HPETEs) and more stable hydroxyeicosatetraenoic acids (HTETEs) typically lead to vascular smooth muscle cell migration. These lipoxygenase products activate many of the pathway-links to increased vascular and renal disease, including protein kinase C (PKC), oncogene activation, and increased matrix production. In particular, these lipoxygenase enzymes generate superoxide radicals via oxidation in pyridine nucleotides. A series of free radical catalyzed peroxidation products of arachidonic acid, called isoprostanes, is formed in a cyclo-oxygenase-independent manner and remains associated with membrane phospholipids until released by phospholipases. Defense mechanisms are critically important for the ultimate effect of oxidative stress and free radicals on cells and tissues within the body. Such defense mechanisms typically interrupt lipid peroxidation and inorganic free radical reaction or scavenge the reactive intermediates formed.

In accordance with the present invention, the dietary supplement described herein is used to scavenge lipid hydroperoxides and superoxide anion free radicals within the body, thereby reducing cellular damage in the human body. The dietary supplement includes a combination of compounds that work at the cellular level to increase the positive functionality of cells in the body, including cell regeneration and cell function. The combination of compounds increases the ability of cells within the body to absorb and utilize nutrients such as vitamins and minerals. The combination has also stimulated the production of T-cells within the immune system. The T-cells are a type of lymphocyte or white blood cell that lead the attack against infections within the body, end the immune response, and/or kill cancer cells and/or cells infected with a virus.

In one embodiment, one fluid ounce (30 mL) of the dietary supplement is consumed per day to reduce toxins produced by natural cell processes with in the human body. Alternatively, other embodiments include the consumption of more than one fluid ounce per day or less than one fluid ounce per day. In a further embodiment, the dietary supplement is taken in the morning and/or before meals when the stomach is typically empty.

Experiments conducted in the research and development lab of Morinda, Inc. have indicated that that regular intake of the dietary supplement, such as Tahitian Noni,® provides a stronger effect to scavenge superoxide anion free radicals within the body than the regular intake of vitamin C, pycnogenol® (maritime pine bark extract), or grape seed powder. In particular, Tahitian Noni® may be used as a dietary supplement to scavenge lipid hydroperoxides and superoxide anion free radicals within the body. Examples of such experiments conducted and results achieved are more fully described in U.S. Provisional Patent Application Serial No. 60/251,417, filed Dec. 5, 2000, entitled “ANTIOXIDANT STUDIES ON TAHITIAN NONI JUICE,” which is incorporated herein by reference.

The results indicate that a dietary supplement having juice from the Morinda citrifolia may be used to scavenge lipid hydroperoxides and/or superoxide anion free radicals. Furthermore, a daily intake of Tahitian Noni® has a stronger effect to scavenge superoxide anion free radicals than Vitamin C, Pycnogenol,® (maritime pine bark extract), or grape seed powder. Furthermore, Tahitian Noni® may be consumed together with Vitamin C, Pycnolgeol,® (maritime pine bark extract), and/or grape seed powder to scavenge lipid hydroperoxides and/or superoxide anion free radical. Moreover, the utilization of the dietary supplement, such as Tahitian Noni,® in accordance with the present invention allows an individual to escape or at least delay the onset of inherited diseases and age-associated declines in vision, hearing, and memory loss and other age-associated physiological declines.

Thus, as discussed herein, the embodiments of the present invention embrace reducing cellular damage in the human body. In particular, the present invention relates to the use of a dietary supplement to scavenge lipid hydroperoxides and superoxide anion free radicals within the body.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. A method for reducing cellular damage, the method comprising the step for providing a dietary supplement for consumption to reduce cellular damage, wherein the dietary supplement includes juice from fruit of a Morinda citrifolia.

2. A method as recited in claim 1, further comprising the step for providing a recommended use of the dietary supplement, wherein the recommended use includes one of:
(i) consuming one fluid ounce of the dietary supplement per day;
(ii) consuming more than one fluid ounce of the dietary supplement per day; and
(iii) consuming less than one fluid ounce of the dietary supplement per day.

3. A method as recited in claim 2, wherein the recommended use further includes consuming the dietary supplement before a meal.

4. A method as recited in claim 1, wherein the step for providing includes the steps for:
harvesting the fruit from the Morinda citrifolia;
preparing the harvested fruit for processing;
processing the prepared fruit to obtain the dietary supplement; and
packaging the dietary supplement.

5. A method as recited in claim 4, wherein the fruit is harvested when it is at least one inch long and up to twelve inches in diameter.

6. A method as recited in claim 4, wherein the step for preparing the harvested fruit includes at least one of the steps for:
(i) cleaning the harvested fruit;
(ii) allowing the harvested fruit to ripen;
(iii) eliminating spoiled fruit from the harvested fruit; and
(iv) placing the harvested fruit in one or more plastic lined containers.

7. A method as recited in claim 4, wherein the step for processing the prepared fruit includes the steps for:
separating the juice of the harvested fruit from at least one of:
(i) seeds of the harvested fruit;
(ii) peel of the harvested fruit; and
(iii) pulp of the harvested fruit; mixing the juice of the harvested fruit with at least one of:
(i) a flavoring;
(ii) a sweetener;
(iii) a nutritional ingredient;
(iv) a botanical extract; and
(v) a coloring; and
heating the dietary supplement to a temperature of at least 181° F.

8. A method as recited in claim 4, wherein the step for packaging the dietary supplement includes the step for packaging the dietary supplement into a container that comprises at least one of:
(i) glass; and
(ii) plastic.

9. A method for scavenging lipid hydroperoxides and superoxide anion free radicals within a body, the method comprising the steps for:
providing a dietary supplement for consumption to perform the scavenging of lipid hydroperoxides and superoxide anion free radicals within the body, wherein the dietary supplement includes juice from fruit of a Morinda citrifolia; and
providing a recommended use of the dietary supplement.

10. A method as recited in claim 9, wherein the recommended use includes one of:
(i) consuming one fluid ounce of the dietary supplement per day;
(ii) consuming more than one fluid ounce of the dietary supplement per day; and
(iii) consuming less than one fluid ounce of the dietary supplement per day.

11. A method as recited in claim 10, wherein the recommended use further includes consuming the dietary supplement before a meal.

12. A method as recited in claim 9, wherein the dietary supplement provided includes reconstituted Morinda citrifolia fruit juice from pure juice puree of French Polynesia.

13. A dietary supplement comprising:
juice harvested from a Morinda citrifolia; and
at least one of:
(i) a flavoring;
(ii) a sweetener;
(iii) a nutritional ingredient;
(iv) a botanical extract; and
(v) a coloring.

14. A dietary supplement as recited in claim 13, wherein the juice is pure juice puree, and wherein the Morinda citrifolia is from French Polynesia.

15. A dietary supplement as recited in claim 13, wherein the dietary supplement scavenges one or more lipid hydroperoxides.

16. A dietary supplement as recited in claim 13, wherein the dietary supplement scavenges one or more superoxide anion free radicals.

17. A dietary supplement as recited in claim 13, wherein the dietary supplement increases cell functionality.

18. A dietary supplement as recited in claim 17, wherein cell functionality includes at least one of:
(i) cell regeneration;
(ii) an ability to absorb nutrients; and
(iii) production of one or more T-cells.

19. A dietary supplement as recited in claim 13, further comprising at least one of:
(i) Vitamin C;
(ii) maritime pine bark extract; and
(iii) grape seed powder.

20. A dietary supplement as recited in claim 13, wherein the dietary supplement delays the onset of one or more inherited diseases.

21. A dietary supplement as recited in claim 20, wherein the inherited diseases include at least one of:
(i) Alzheimer's disease;
(ii) cancer;
(iii) diabetes;
(iv) heart disease;
(v) macular degeneration; and
(vi) Parkinson's disease.

22. A dietary supplement as recited in claim 13, wherein the dietary supplement delays the onset of one or more age-associated declines.

23. A dietary supplement as recited in claim 22, wherein the age-associated declines include at least one of:
(i) vision loss;
(ii) hearing loss; and
(iii) memory loss.
